(19) United States
(12) Patent Application Publication
(10) Pub. No.: US 2019/0271098 A1
(43) Pub. Date: Sep. 5, 2019

(54) FLEXIBLE ELECTROSPUN FIBER RODS AND METHODS OF MANUFACTURE

(71) Applicant: NANOFIBER SOLUTIONS, LLC, Hilliard, OH (US)

(72) Inventors: Jed K. JOHNSON, London, OH (US); Devan Ohst, Columbus, OH (US); Michael Helterbran, Cable, OH (US)

(73) Assignee: NANOFIBER SOLUTIONS, LLC, Hilliard, OH (US)

(21) Appl. No.: 16/291,783

(22) Filed: Mar. 4, 2019

Related U.S. Application Data
(63) Continuation of application No. 62/637,635, filed on Mar. 2, 2018.

(51) Int. Cl. D01D 5/00; A61B 17/70; A61L 27/18

(52) U.S. Cl. CPC D01D 5/0015; A61L 27/18; A61B 17/7019

(57) ABSTRACT

The instant disclosure is directed to flexible electrospun fiber rods and methods of manufacturing such rods. A scaffold may comprise a flexible rod having a spiral cross-section, the flexible rod comprising electrospun polymer fibers. The electrospun polymer fibers may be substantially aligned or randomly oriented with respect to one another. The flexible rod may further comprise substantially uniformly distributed pores. The flexible rod may have a length and a diameter of a native mammalian tendon or ligament. A method of manufacturing such a scaffold may comprise forming a layer of polymer fibers on a mandrel by electrospinning, rolling the layer from a first end of the mandrel to a second end of the mandrel to form a toroid at the second end of the mandrel, and cutting the toroid off the mandrel to form a flexible rod having a spiral cross-section.

### CROSS-REFERENCE TO RELATED APPLICATIONS

This application claims priority to and benefit of U.S. Provisional Application Ser. No. 62/637,635, filed Mar. 2, 2018, entitled “Flexible Electrospun Fiber Rods and Methods of Manufacture,” which is incorporated herein by reference in its entirety.

### BACKGROUND

It is increasingly common for tendons and ligaments to be surgically repaired or replaced. The current standard for repairing an anterior cruciate ligament (ACL), for example, is to harvest one or more tendons or ligaments from elsewhere in the body and using them to replace the torn or ruptured ACL. Another common approach for ACL repair in animals is a tibial plateau-leveling osteotomy (TPLO), in which a specialized plate is affixed to the tibia. Such a repair frequently limits range of motion in the affected joint, and has long-term issues associated with stress shielding and a change of mechanical loading, often resulting in replacement of the contralateral ACL. Yet another approach to replace a tendon or ligament involves placing a strong suture material within bone tunnels and fixing the suture under tension to serve as a mechanical substitute for the native tendon or ligament. However, the suture material often leads to poor integration with bone, resulting in concerns about long-term viability of such a technique. Thus, there exists a need for a flexible tendon or ligament replacement that can encourage bone integration.

### SUMMARY

The instant disclosure is directed to flexible electrospun fiber rods, and methods of manufacturing such rods. In one embodiment, a scaffold may comprise a flexible rod having a spiral cross-section, the flexible rod comprising electrospun polymer fibers. In some embodiments, the electrospun polymer fibers may be substantially aligned with respect to one another; in other embodiments, the electrospun polymer fibers may be randomly oriented with respect to one another. In certain embodiments, the flexible rod may further comprise substantially uniformly distributed pores. In some embodiments, the flexible rod may have a length and a diameter of a native mammalian tendon or ligament.

In one embodiment, a method of manufacturing a scaffold may comprise forming a layer of polymer fibers on a mandrel by electrospinning, rolling the layer from a first end of the mandrel to a second end of the mandrel to form a toroid at the second end of the mandrel, and cutting the toroid off the mandrel to form a flexible rod having a spiral cross-section. In some embodiments, the polymer fibers may be substantially aligned with respect to one another; in other embodiments, the polymer fibers may be randomly oriented with respect to one another. In certain embodiments, the flexible rod may further comprise substantially uniformly distributed pores. In some embodiments, the flexible rod may have a length and a diameter of a native mammalian tendon or ligament.

In an embodiment, a scaffold may be formed by a process comprising forming a layer of polymer fibers on a mandrel by electrospinning, rolling the layer from a first end of the mandrel to a second end of the mandrel to form a toroid at the second end of the mandrel, and cutting the toroid off the mandrel to form a flexible rod having a spiral cross-section. Further embodiments of the instant disclosure are described herein.

### BRIEF DESCRIPTION OF THE DRAWINGS

FIG. 1A is a scanning electron microscope (SEM) image of a scaffold produced by a conventional method of manufacturing that involves twisting and straining the scaffold, among other things.

FIG. 1B is an SEM image of an alternative view of the scaffold shown in FIG. 1A.

FIG. 2A is an SEM image of an embodiment of a scaffold comprising a flexible rod having a spiral cross-section, in accordance with the present disclosure.

FIG. 2B is an SEM image of a perpendicular cross-section of the scaffold shown in FIG. 2A, which comprises substantially uniformly distributed pores, in accordance with the present disclosure.

FIG. 2C is an SEM image of the scaffold shown in FIG. 2A, which comprises substantially aligned electrospun polymer fibers, in accordance with the present disclosure.

FIG. 3A shows an embodiment of a layer of polymer fibers formed on a mandrel by electro spinning, with a first end of the mandrel in the foreground and a second end of the mandrel in the background, in accordance with the present disclosure.

FIG. 3B shows an embodiment of a layer as shown in FIG. 3A, the layer being rolled from the first end of the mandrel to the second end of the mandrel, in accordance with the present disclosure.

FIG. 3C shows an embodiment of a layer as shown in FIG. 3B, the layer being rolled further from the first end of the mandrel to the second end of the mandrel, in accordance with the present disclosure.

FIG. 3D shows an embodiment of a layer as shown in FIG. 3C, the layer being rolled further from the first end of the mandrel to the second end of the mandrel to form an embodiment of a toroid, in accordance with the present disclosure.

FIG. 4 shows an embodiment of a scaffold being implanted as a replacement for a deep flexor tendon in an equine subject, in accordance with the present disclosure.

FIG. 5 shows an embodiment of a scaffold being implanted as a replacement for a tendon in a bovine subject, in accordance with the present disclosure.

FIG. 6 shows an embodiment of a scaffold being implanted as a replacement for an anterior cruciate ligament in a canine cadaver model, in accordance with the present disclosure.

FIG. 7A is a graph showing the maximum displacement (in mm) at 100,000 cycles of (i) a scaffold implanted as a replacement for an anterior cruciate ligament, and (ii) the native intact contralateral control anterior cruciate ligament in three canine cadaver subjects.

FIG. 7B is a graph showing the displacement (in mm) of a scaffold implanted as a replacement for an anterior cruciate ligament over 100,000 cycles in a representative canine cadaver subject.

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term comprising is used as the transition phrase, such embodiments can also be envisioned with replacement of the term “comprising” with the terms “consisting of” or “consisting essentially of.”

The terms “animal,” “patient,” “mammal,” and “subject” as used herein include, but are not limited to, humans and non-human vertebrates such as wild, domestic, and farm animals. In some embodiments, the terms “animal,” “patient,” “mammal,” and “subject” may refer to humans.

As used herein, the term “biocompatible” refers to non-harmful compatibility with living tissue. Biocompatibility is a broad term that describes a number of materials, including bioinert materials, bioactive materials, bioabsorbable materials, biostable materials, biotolerant materials, or any combination thereof.

### Electrospinning Fibers

Electrospinning is a method which may be used to process a polymer solution into a fiber. In embodiments wherein the diameter of the resulting fiber is on the nanometer scale, the fiber may be referred to as a nanofiber. Fibers may be formed into a variety of shapes by using a range of receiving surfaces, such as mandrels or collectors. In some embodiments, a flat shape, such as a sheet or sheet-like fiber mold, a fiber scaffold and/or tube, or a tubular lattice, may be formed by using a substantially round or cylindrical mandrel. In certain embodiments, the electrospun fibers may be cut and/or unrolled from the mandrel as a fiber mold to form the sheet. The resulting fiber molds or shapes may be used in many applications, including the repair or replacement of biological structures. In some embodiments, the resulting fiber scaffold may be implanted into a biological organism or a portion thereof.

Electrospinning methods may involve spinning a fiber from a polymer solution by applying a high DC voltage potential between a polymer injection system and a mandrel. In some embodiments, one or more charges may be applied to one or more components of an electrospinning system. In some embodiments, a charge may be applied to the mandrel, the polymer injection system, or combinations or portions thereof. Without wishing to be bound by theory, as the polymer solution is ejected from the polymer injection system, it is thought to be destabilized due to its exposure to a charge. The destabilized solution may then be attracted to a charged mandrel. As the destabilized solution moves from the polymer injection system to the mandrel, its solvents may evaporate and the polymer may stretch, leaving a long, thin fiber that is deposited onto the mandrel. The polymer solution may form a Taylor cone as it is ejected from the polymer injection system and exposed to a charge.

In certain embodiments, a first polymer solution comprising a first polymer and a second polymer solution comprising a second polymer may each be used in a separate polymer injection system at substantially the same time to produce one or more electrospun fibers comprising the first polymer interspersed with one or more electrospun fibers comprising the second polymer. Such a process may be referred to as “co-spinning” or “co-electrospinning,” and a scaffold produced by such a process may be described as a co-spun or co-electrospun scaffold.

### Polymer Injection System

A polymer injection system may include any system configured to eject some amount of a polymer solution into an atmosphere to permit the flow of the polymer solution from the injection system to the mandrel. In some embodiments, the polymer injection system may deliver a continuous or linear stream with a controlled volumetric flow rate of a polymer solution to be formed into a fiber. In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with each syringe containing a different polymer solution. In some embodiments, a charge may be applied to the polymer injection system, or to a portion thereof. In some embodiments, a charge may be applied to a needle or needle-like component of the polymer injection system.

In some embodiments, the polymer solution may be ejected from the polymer injection system at a flow rate of less than or equal to about 5 mL/h per needle. In other embodiments, the polymer solution may be ejected from the polymer injection system at a flow rate per needle in a range from about 0.01 mL/h to about 50 mL/h. The flow rate at which the polymer solution is ejected from the polymer injection system per needle may be, in some non-limiting examples, about 0.01 mL/h, about 0.05 mL/h, about 0.1 mL/h, about 0.5 mL/h, about 1 mL/h, about 2 mL/h, about 3 mL/h, about 4 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any range between any two of these values, including endpoints.

As the polymer solution travels from the polymer injection system toward the mandrel, the diameter of the resulting fibers may be in the range of about 0.1 μm to about 10 μm. Some non-limiting examples of electrospun fiber diameters may include about 0.1 μm, about 0.2 μm, about 0.25 μm, about 0.5 μm, about 1 μm, about 2 μm, about 5 μm, about 10 μm, about 20 μm, or ranges between any two of these values, including endpoints. In some embodiments, the electrospun fiber diameter may be from about 0.25 μm to about 20 μm.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include, for example, non-resorbable polymers, resorbable polymers, natural polymers, or a combination thereof.

In some embodiments, the polymers may include, for example, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate), trimethylene carbonate, polydiols, polyesters, collagen, gelatin, fibrin, fibronectin, albumin, hyaluronic acid, elastin, chitosan, alginate, silk, copolymers thereof, and combinations thereof.

It may be understood that polymer solutions may also include a combination of one or more of non-resorbable, resorbable polymers, and naturally occurring polymers in any combination or compositional ratio. In an alternative embodiment, the polymer solutions may include a combination of two or more non-resorbable polymers, two or more resorbable polymers or two or more naturally occurring polymers. In some non-limiting examples, the polymer solution may comprise a weight percent ratio of, for example, from about 5% to about 90%. Non-limiting examples of such weight percent ratios may include about 5%, about 10%, about 15%, about 20%, about 25%, about 30%, about 33%, about 35%, about 40%, about 45%, about 50%, about 55%, about 60%, about 66%, about 70%, about 75%, about 80%, about 85%, about 90%, or ranges between any two of these values, including endpoints.

In some embodiments, the polymer solution may comprise one or more solvents. In some embodiments, the solvent may comprise, for example, acetone, dimethylformamide, dimethylsulfoxide, N-methylpyrrolidone, N,N-dimethylformamide, Nacetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

In some embodiments, the polymer solution may also include additional materials. Non-limiting examples of such additional materials may include radiation opaque materials, contrast agents, electrically conductive materials, fluorescent materials, luminescent materials, antibiotics, growth factors, vitamins, cytokines, steroids, anti-inflammatory drugs, small molecules, sugars, salts, peptides, proteins, cell factors, DNA, RNA, other materials to aid in non-invasive imaging, or any combination thereof. In some embodiments, the radiation opaque materials may include, for example, barium, tantalum, tungsten, iodine, gadolinium, gold, platinum, bismuth, or bismuth (III) oxide. In some embodiments, the electrically conductive materials may include, for example, gold, silver, iron, or polyaniline.

In some embodiments, the additional materials may be present in the polymer solution in an amount from about 1 wt % to about 1500 wt % of the polymer mass. In some non-limiting examples, the additional materials may be present in the polymer solution in an amount of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 100 wt %, about 125 wt %, about 150 wt %, about 175 wt %, about 200 wt %, about 225 wt %, about 250 wt %, about 275 wt %, about 300 wt %, about 325 wt %, about 350 wt %, about 375 wt %, about 400 wt %, about 425 wt %, about 450 wt %, about 475 wt %, about 500 wt %, about 525 wt %, about 550 wt %, about 575 wt %, about 600 wt %, about 625 wt %, about 650 wt %, about 675 wt %, about 700 wt %, about 725 wt %, about 750 wt %, about 775 wt %, about 800 wt %, about 825 wt %, about 850 wt %, about 875 wt %, about 900 wt %, about 925 wt %, about 950 wt %, about 975 wt %, about 1000 wt %, about 1025 wt %, about 1050 wt %, about 1075 wt %, about 1100 wt %, about 1125 wt %, about 1150 wt %, about 1175 wt %, about 1200 wt %, about 1225 wt %, about 1250 wt %, about 1275 wt %, about 1300 wt %, about 1325 wt %, about 1350 wt %, about 1375 wt %, about 1400 wt %, about 1425 wt %, about 1450 wt %, about 1475 wt %, about 1500 wt %, or any range between any of these two values, including endpoints. In one embodiment, the polymer solution may include tantalum present in an amount of about 10 wt % to about 1,500 wt %.

The type of polymer in the polymer solution may determine the characteristics of the electrospun fiber. Some fibers may be composed of polymers that are bio-stable and not absorbable or biodegradable when implanted. Such fibers may remain generally chemically unchanged for the length of time in which they remain implanted. Alternatively, fibers may be composed of polymers that may be absorbed or bio-degraded over time. Such fibers may act as an initial template or scaffold during a healing process. These templates or scaffolds may degrade in vivo once the tissues have a degree of healing by natural structures and cells. It may be further understood that a polymer solution and its resulting electrospun fiber(s) may be composed or more than one type of polymer, and that each polymer therein may have a specific characteristic, such as bio-stability, biodegradability, or bioabsorbability.

### Applying Charges to Electrospinning Components

In an electrospinning system, one or more charges may be applied to one or more components, or portions of components, such as, for example, a mandrel or a polymer injection system, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to mandrel, or portions thereof. In some embodiments, a negative charge may be applied to the mandrel, or portions thereof. In some embodiments, the mandrel, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about −15 kV to about 30 kV, including endpoints. In some non-limiting examples, the charge applied to any component of the electrospinning system, or portions thereof, may be about −15 kV, about −10 kV, about −5 kV, about −4 kV, about −3 kV, about −1 kV, about −0.01 kV, about 0.01 kV, about 1 kV, about 5 kV, about 10 kV, about 11 kV, about 11.1 kV, about 12 kV, about 15 kV, about 20 kV, about 25 kV, about 30 kV, or any range between any two of these values, including endpoints. In some embodiments, any component of the electrospinning system, or portions thereof, may be grounded.

### Mandrel Movement During Electrospinning

During electrospinning, in some embodiments, the mandrel may move with respect to the polymer injection system. In some embodiments, the polymer injection system may move with respect to the mandrel. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the mandrel may be in contact with or mounted upon a support structure that may be moved using one or more motors or motion control systems. The pattern of the electrospun fiber deposited on the mandrel may depend upon the one or more motions of the mandrel with respect to the polymer injection system. In some embodiments, the mandrel surface may be configured to rotate about its long axis. In one non-limiting example, a mandrel having a rotation rate about its long axis that is faster than a translation rate along a linear axis, may result in a nearly helical deposition of an electrospun fiber, forming windings about the mandrel. In another example, a mandrel having a translation rate along a linear axis that is faster than a rotation rate about a rotational axis, may result in a roughly linear deposition of an electrospun fiber along a liner extent of the mandrel.

### Flexible Electrospun Fiber Rods and Methods of Manufacture

The instant disclosure is directed to flexible electrospun fiber rods and methods of manufacturing such rods. It may be understood that the devices and methods described herein may be applied to any soft tissue, connective tissue, tendon, or ligament, and that the examples described herein are non-limiting.

It is increasingly common for tendons and ligaments to be surgically repaired or replaced. Some surgical techniques seek to replace a tendon or ligament by harvesting a less critical tendon or ligament from elsewhere in the body, and using it to replace the torn or ruptured tendon or ligament. Other techniques employ synthetic graft materials. One approach to replace a tendon or ligament involves placing a strong suture material within bone tunnels and fixing the suture under tension to serve as a mechanical substitute for the native tendon or ligament. However, the suture material often leads to poor integration with bone, resulting in concerns about long-term viability of such a technique. Thus, there exists a need for a flexible, easily manipulated tendon or ligament replacement that can encourage bone integration.

One such replacement may include a scaffold comprising a flexible rod having a spiral cross-section, the flexible rod comprising substantially aligned electrospun polymer fibers and substantially uniformly distributed pores. Without wishing to be bound by theory, such a scaffold may provide a high surface area interface with the bone to which it is anchored, and the interface may encourage cell and tissue adhesion and remodeling, ultimately resulting in an improved tendon or ligament replacement. The electrospun polymer fibers of such a scaffold may allow for a high degree of flexibility, allowing the scaffold to be easily manipulated during placement and usage. Furthermore, the ease with which a scaffold may be manipulated may allow it to be used with existing surgical techniques, improving surgeons' comfort with such a scaffold. In embodiments where such electrospun polymer fibers are substantially aligned with respect to one another, the fiber alignment may provide high levels of strength, allowing the scaffold to function as a tendon or ligament replacement immediately after implantation. Furthermore, the degree of stiffness or elasticity of the scaffold may be tailored by adjusting the degree of fiber alignment.

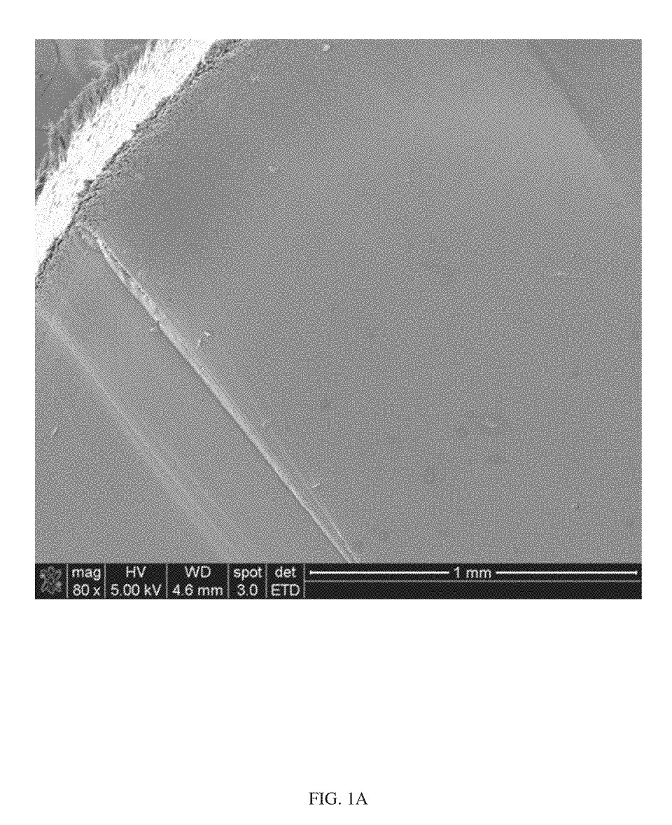

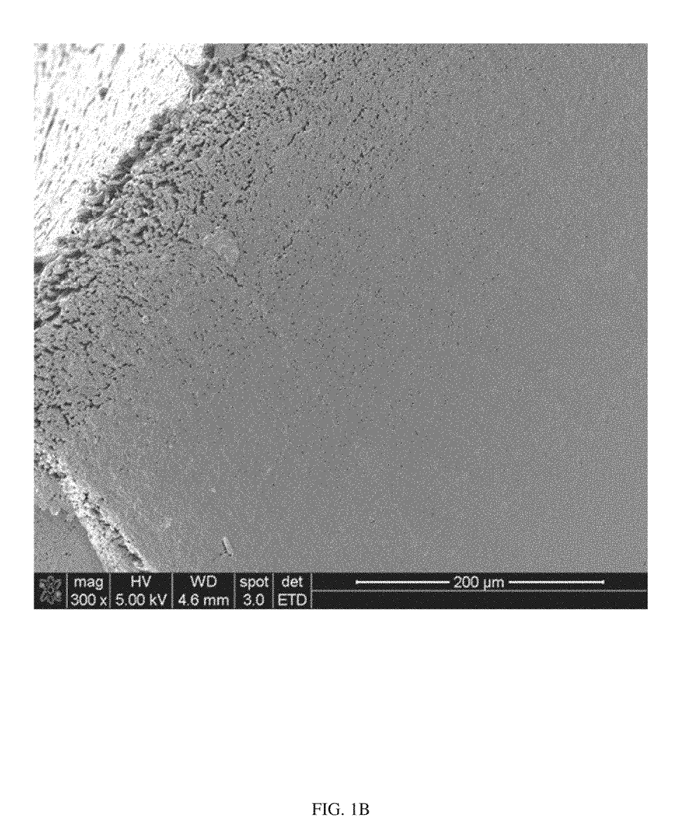

Conventional methods of manufacturing synthetic scaffolds result in poorly formed scaffolds with non-uniform porosity and non-uniform mechanical properties. FIG. 1A and FIG. 1B are scanning electron microscope (SEM) images of a scaffold produced by a conventional method of manufacturing that involves twisting and straining the scaffold, among other things. The resulting conventional scaffold features, among other things, a substantially solid, pore-free core, and a porous perimeter, both of which lead to sub-optimal mechanical properties and poor cellular infiltration. In contrast, the scaffolds disclosed herein may have, in some embodiments, substantially uniformly distributed pores and electrospun fibers that are substantially aligned with respect to one another, leading to improved mechanical properties and cellular infiltration.

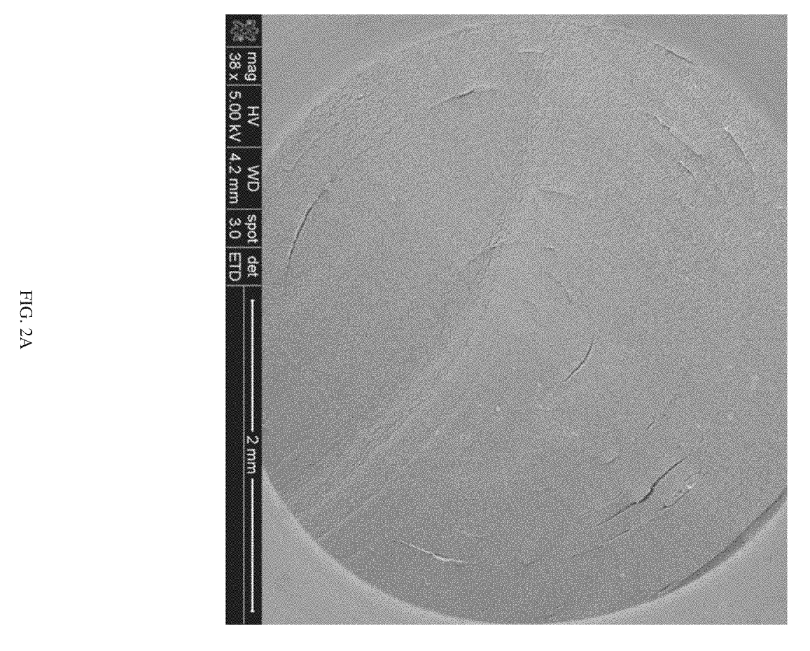

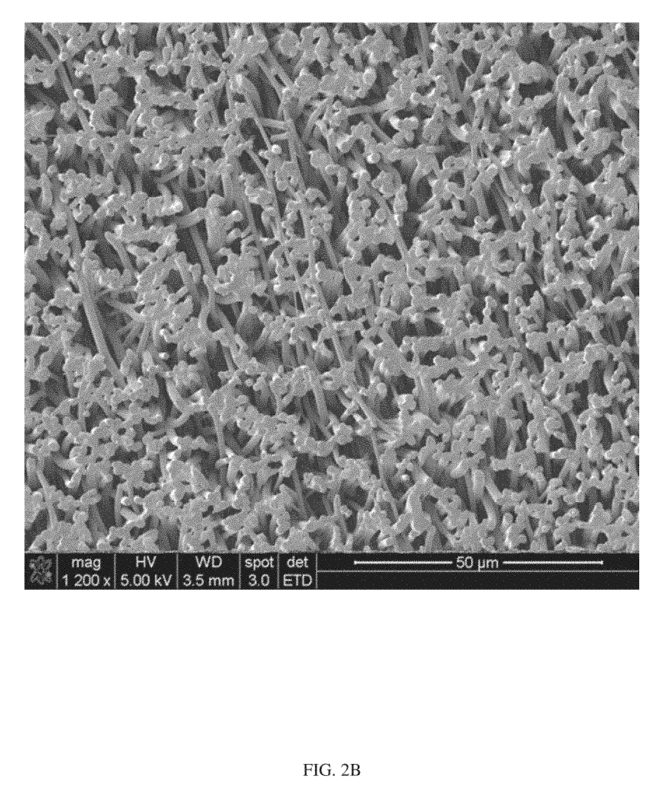

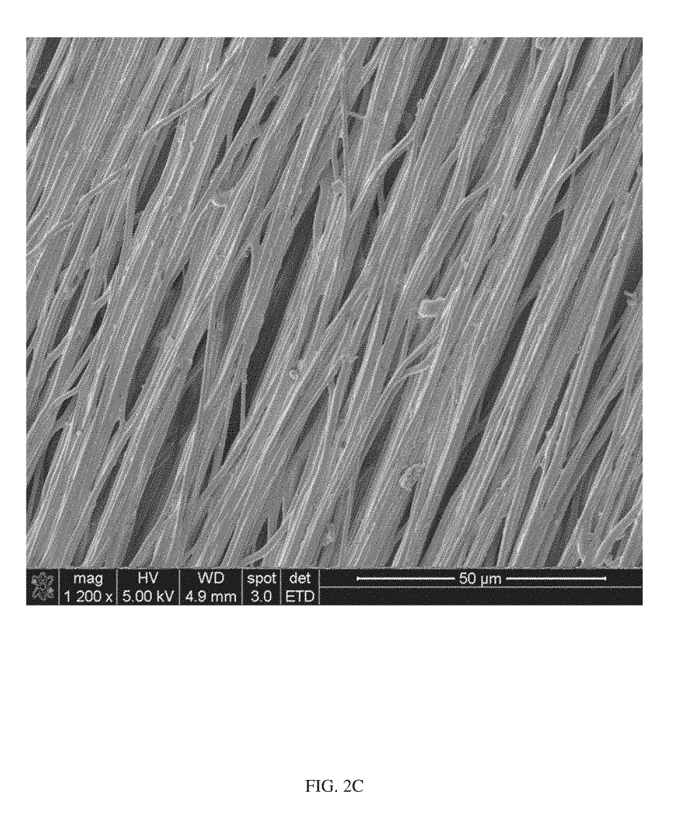

In one embodiment, a scaffold may comprise a flexible rod having a spiral cross-section, the flexible rod comprising electrospun polymer fibers. FIG. 2A is an SEM image of an embodiment of a scaffold comprising a flexible rod having a spiral cross-section. In certain embodiments, the spiral cross-section may appear to have multiple concentric layers of electrospun fibers.

In some embodiments, the electrospun polymer fibers may comprise a polymer such as, for example, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, copolymers thereof, and combinations thereof. In certain embodiments, the electrospun polymer fibers may comprise at least two electrospun polymer fibers, each electrospun polymer comprising a polymer independently selected from the examples above, wherein the at least two electrospun polymer fibers are co-electrospun. In one embodiment, the electrospun polymer fibers may comprise co-electrospun polyethylene terephthalate fibers and polyurethane fibers. In certain embodiments, the polyethylene terephthalate fibers and polyurethane fibers may be present in the scaffold in a weight ratio of about 2:8. In other embodiments, the electrospun polymer fibers may be electrospun from a single polymer solution comprising more than one polymer (i.e. not co-electrospun). In one such embodiment, the electrospun polymer fibers may comprise a blend of polyethylene terephthalate and polyurethane. In certain embodiments, the polyethylene terephthalate and polyurethane may be present in the electrospun polymer fibers in a weight ratio of about 2:8.

In some embodiments, the electrospun polymer fibers may be randomly oriented with respect to one another. In other embodiments, the electrospun polymer fibers may be aligned or substantially aligned with respect to one another, such that the fibers are parallel or substantially parallel to one another. In still other embodiments, the electrospun polymer fibers may be a combination of randomly oriented and aligned or substantially aligned with respect to one another. In an embodiment, a combination of randomly oriented or and aligned or substantially aligned electrospun polymer fibers may be used to tailor the stiffness and/or elasticity of the flexible rod. FIG. 2C is an SEM image of an embodiment of a flexible rod comprising substantially aligned electro spun polymer fibers.

In certain embodiments, the flexible rod may further comprise substantially uniformly distributed pores. FIG. 2B is an SEM image of an embodiment of a flexible rod comprising substantially uniformly distributed pores. In certain embodiments, the flexible rod may comprise substantially uniformly distributed pores throughout the thickness and length of the flexible rod.

In some embodiments, the flexible rod may have a diameter from about 1 mm to about 25 mm. In certain embodiments, the diameter of the flexible rod may be uniform throughout the length of the rod, while in other embodiments the diameter of the flexible rod may vary with the length of the rod. In some embodiments, the diameter of the flexible rod may be, for example, about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, or any range between any two of these values, including endpoints. In one embodiment, the flexible rod may have a diameter of about 4.2 mm.

In some embodiments, the flexible rod may have a length from about 1 cm to about 50 cm. The length of the flexible rod may be, for example, about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, or any range between any two of these values, including endpoints. In one embodiment, the flexible rod may have a length of about 6 cm.

In some embodiments, the flexible rod may have a length and a diameter of a native mammalian tendon or ligament. The tendon or ligament may be, for example, a supraspinatus tendon, an infraspinatus tendon, a subscapularis tendon, a deltoid tendon, a biceps tendon, a triceps tendon, an anterior cruciate ligament, a posterior cruciate ligament, a medial collateral ligament, a lateral collateral ligament, an illiotibial band, a quadriceps tendon, a hamstring tendon, a sartorius tendon, an Achilles tendon, a tibialis anterior tendon, or combinations thereof. In some embodiments, the mammalian tendon or ligament may be from, for example, a dog, a cow, a horse, a sheep, a goat, a human, or any other mammal for which tendon or ligament repair or replacement may be an appropriate surgical approach.

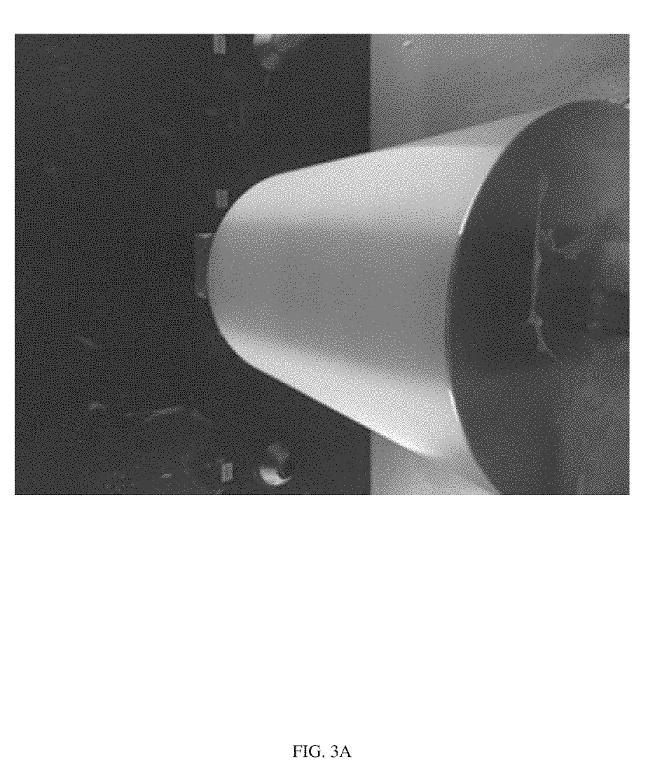

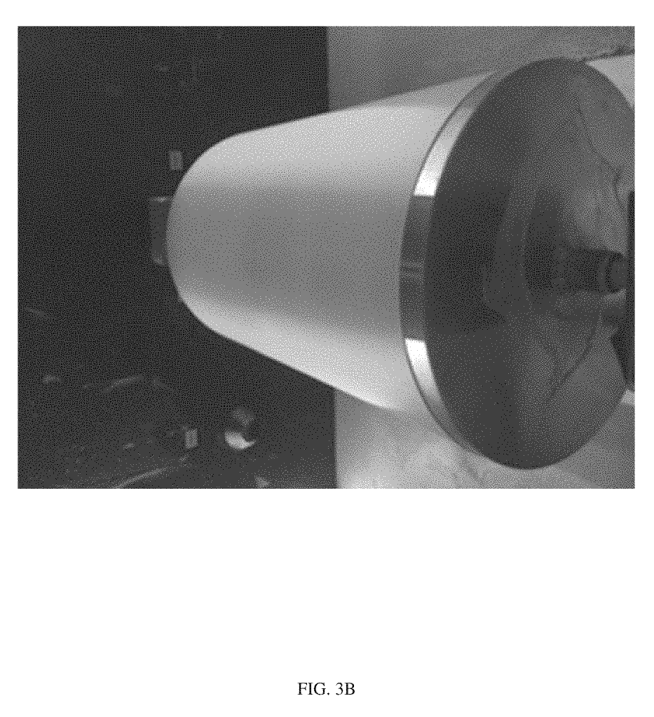

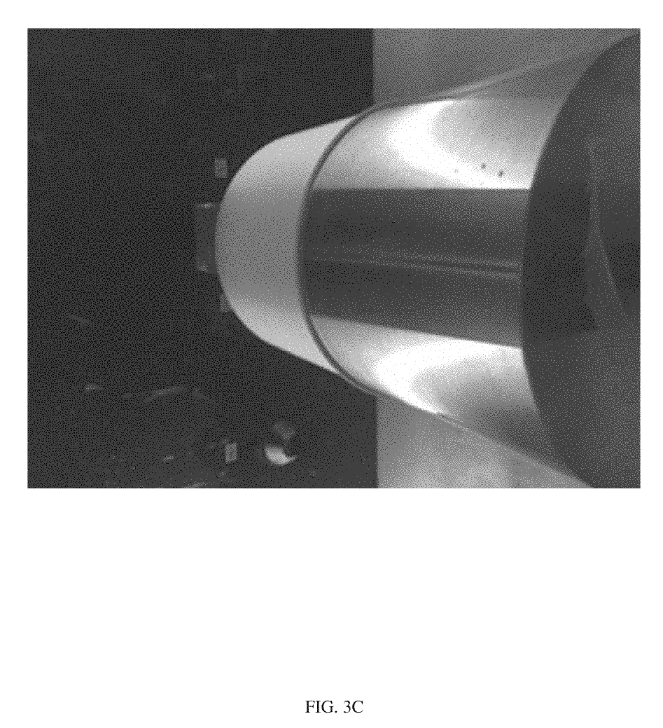

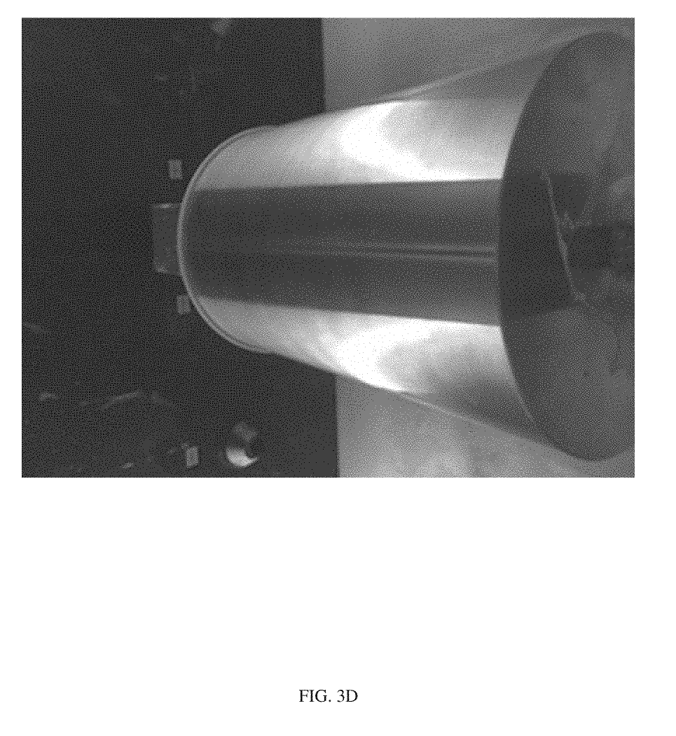

In one embodiment, a method of manufacturing a scaffold may comprise forming a layer of polymer fibers on a mandrel by electrospinning, as described herein. In certain embodiments, the mandrel may be a cylindrical mandrel, as shown in FIG. 3A, FIG. 3B, FIG. 3C, and FIG. 3D. FIG. 3A shown an embodiment of a layer of polymer fibers formed on a mandrel by electrospinning, with a first end of the mandrel in the foreground and a second end of the mandrel in the background.

In certain embodiments, the layer of polymer fibers formed on the mandrel by electrospinning may have a thickness from about 10 μm to about 1,000 μm. The thickness of the layer may be, for example, about 10 μm, about 20 μm, about 30 μm, about 40 μm, about 50 μm, about 60 μm, about 70 μm, about 80 μm, about 90 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 525 μm, about 550 μm, about 575 μm, about 600 μm, about 625 μm, about 650 μm, about 675 μm, about 700 μm, about 725 μm, about 750 μm, about 775 μm, about 800 μm, about 825 μm, about 850 μm, about 875 μm, about 900 μm, about 925 μm, about 950 μm, about 975 μm, about 1,000 μm, or any range between any two of these values, including endpoints. In some embodiments, forming a layer of polymer fibers on a mandrel by electrospinning may comprise co-electrospinning fibers comprising two or more independently selected polymers, as described herein. In other embodiments, forming a layer of polymer fibers on a mandrel by electrospinning may comprise electrospinning a polymer solution comprising a blend of two or more independently selected polymers, as described herein. In some embodiments, electrospinning a polymer solution comprising a blend of two or more polymers may result in monolithic electrospun polymer fibers comprising the two or more polymers. In certain embodiments, the layer of polymer fibers may comprise two or more layers of polymer fibers.

In some embodiments, the method of manufacturing the scaffold may further comprise rolling the layer from a first end of the mandrel to a second end of the mandrel to form a toroid at the second end of the mandrel. Without wishing to be bound by theory, the process of rolling the layer from the first end of the mandrel to the second end of the mandrel may allow for the formation of a toroid at the second end of the mandrel without substantially altering the alignment of the electrospun polymer fibers or the substantially uniform distribution of the pores throughout the thickness of the toroid. Furthermore, the particular polymers comprised in the electrospun polymer fibers may allow for optimal mechanical properties to allow the step of rolling to occur with minimal, if any, damage to the electrospun polymer fibers themselves. FIG. 3B shows an embodiment of a layer as shown in FIG. 3A, the layer being rolled from the first end of the mandrel to the second end of the mandrel. FIG. 3C shows the layer of FIG. 3B being rolled further from the first end of the mandrel to the second end of the mandrel. Finally, FIG. 3D shows the layer shown in FIGS. 3A, 3B, and 3C being rolled further from the first end of the mandrel to the second end of the mandrel to form an embodiment of a toroid. Taken together, FIG. 3A, FIG. 3B, FIG. 3C, and FIG. 3D show an embodiment of the step of rolling the layer from a first end of the mandrel to a second end of the mandrel to form a toroid at the second end of the mandrel.

In some embodiments, the toroid may have a diameter from about 1 cm to about 100 cm. The diameter of the toroid may be, for example, about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, about 100 cm, or any range between any two of these values, including endpoints. In one embodiment, the toroid may have a diameter of about 20 cm.

In some embodiments, the method of manufacturing the scaffold may further comprise cutting the toroid off the mandrel to form a flexible rod having a spiral cross-section. In certain embodiments, the spiral cross-section of the flexible rod may be a result of the step of rolling the layer from a first end of the mandrel to a second end of the mandrel to form a toroid at the second end of the mandrel. The flexible rod that results from the step of cutting the toroid off the mandrel may have any of the properties or features of flexible rods as described herein.

In an embodiment, a scaffold as described herein may be formed by a process comprising forming a layer of polymer fibers on a mandrel by electrospinning, rolling the layer from a first end of the mandrel to a second end of the mandrel to form a toroid at the second end of the mandrel, and cutting the toroid off the mandrel to form a flexible rod having a spiral cross-section, as described herein.

### EXAMPLES

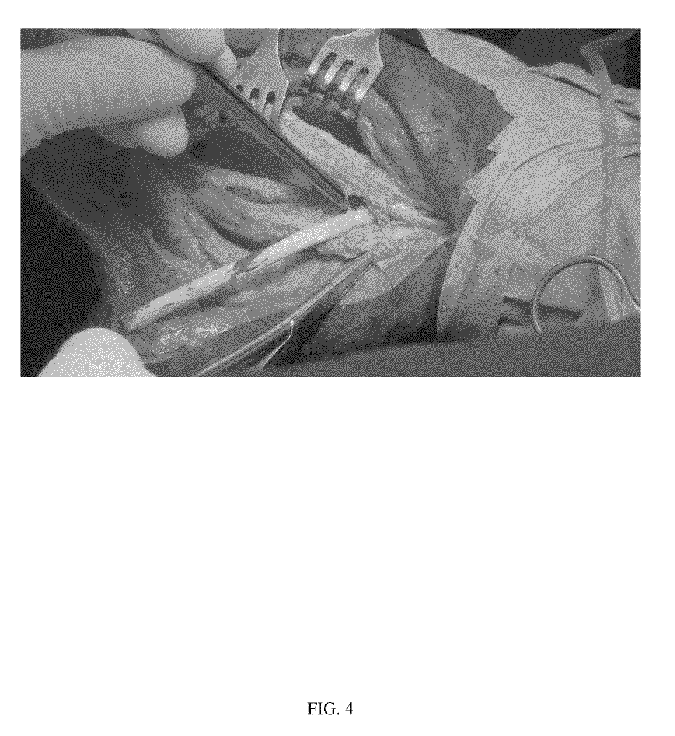

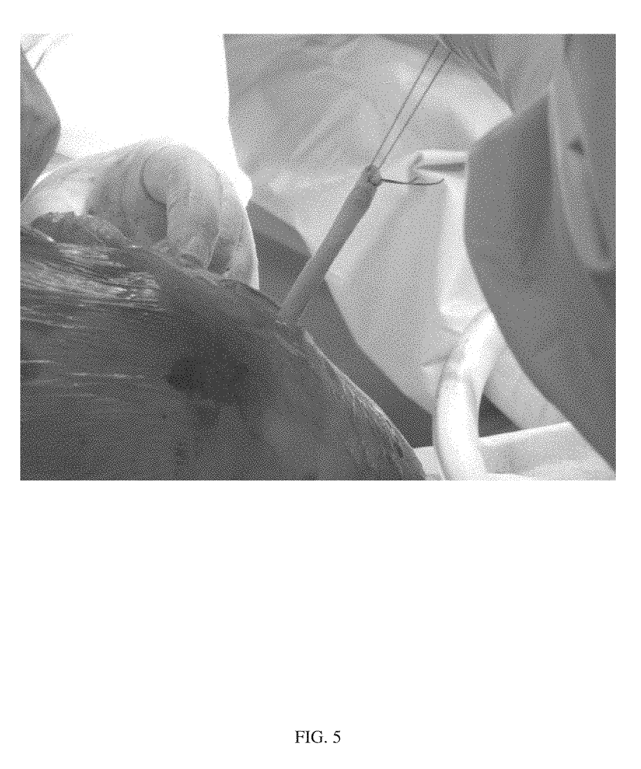

Scaffolds as described herein have been surgically implanted as tendon or ligament replacements in several mammalian subjects. FIG. 4 shows an embodiment of a scaffold as described herein being implanted as a replacement for a deep flexor tendon in an equine subject. Similarly, FIG. 5 shows an embodiment of a scaffold as described herein being implanted as a replacement for a tendon in a bovine subject.

### Example 1

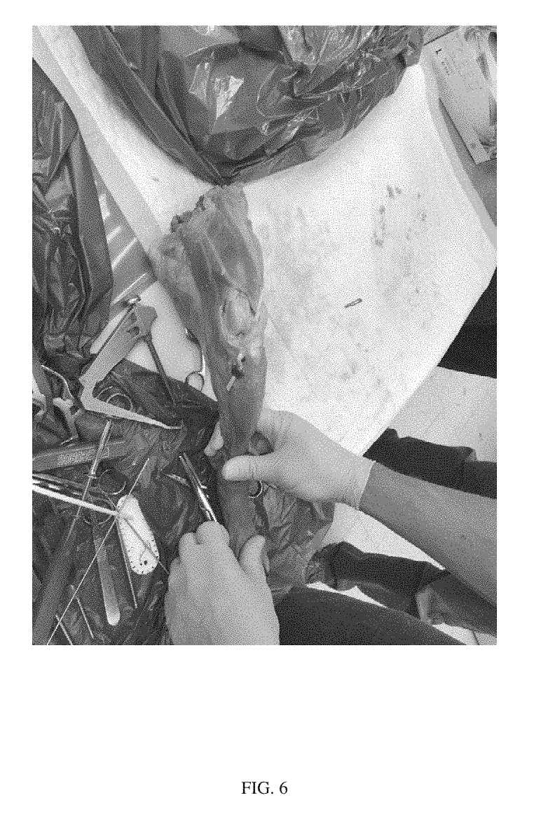

In one example, a scaffold as described herein was placed in a canine cadaver leg as an ACL replacement using two different fixation methods commonly used in ACL repair and fixation. The native canine ACL was removed, and the scaffold was implanted in its place, as shown in FIG. 6. The resulting cadaver joint was mounted into a standard tensile testing construct for destructive tensioning with a 135° angle between the long axes of the tibia and femur. When using dual bone tunnels to fix the scaffold in place, the resulting joint had a peak tensile load of 1060N and 1107N, and a stiffness of 87.2 N/mm and 66.7 N/mm, respectively. When using a bone tunnel and an over-the-top method to fix the scaffold in place, the resulting joint had a peak tensile load of 2128N and 2179, and a stiffness of 104.0 N/mm and 161.7 N/mm, respectively. The reported peak tensile loads are the peak loads recorded before total destructive failure of the canine cadaver joint. The scaffold neither broke nor became dislodged from the fixation points in any of the conducted tests and all failures occurred within the tibia or femur bones at their fixation points within the tensile testing construct.

### Example 2

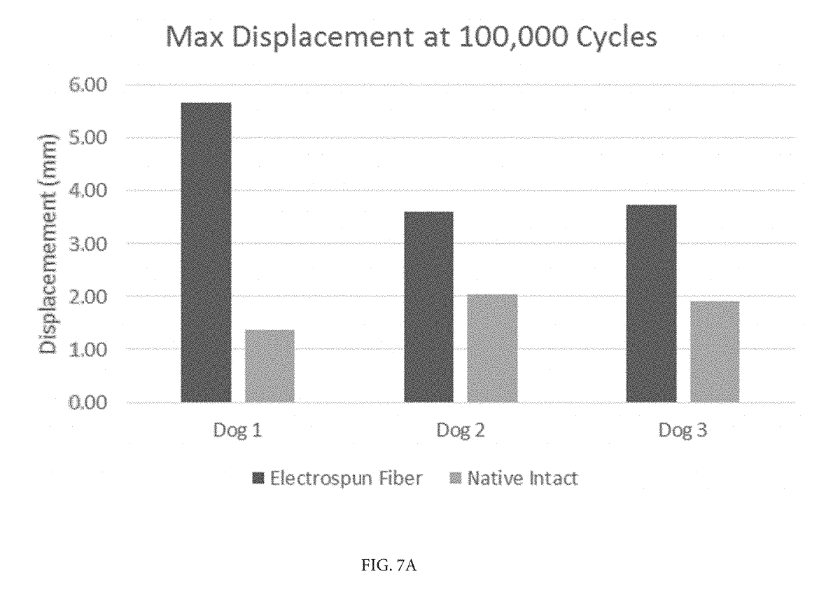

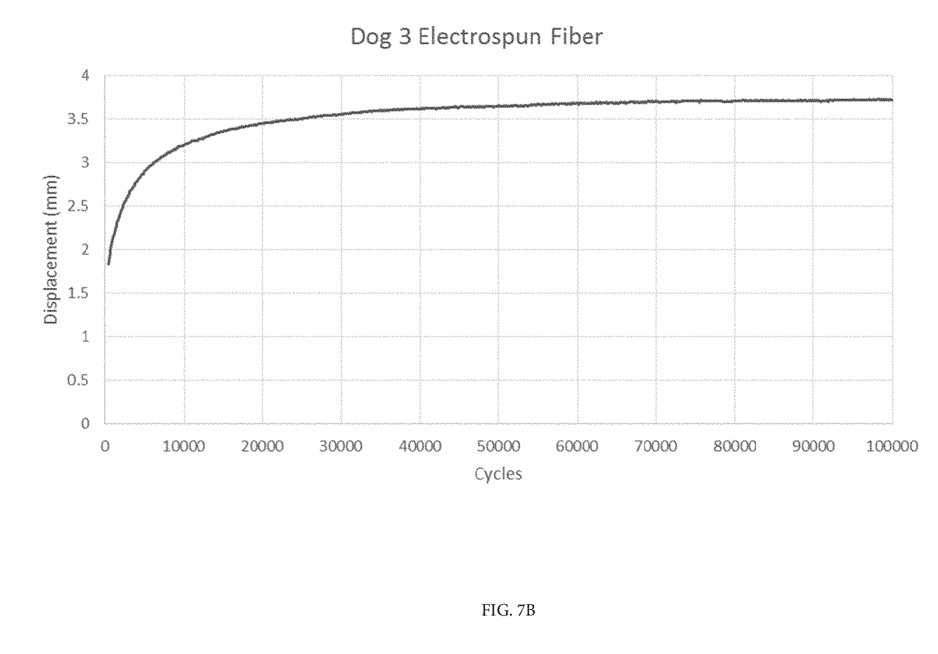

In another example, scaffolds as described herein were placed in three canine cadaver limbs as ACL replacements, as described in Example 1 above. The three canine cadaver limbs and their native intact contralateral controls were then cycled from 8N to 80N load for 100,000 cycles. FIG. 7A shows the maximum displacement (in mm) at 100,000 loading cycles for the scaffold-implanted limbs (“Electrospun Fiber”) compared to the same measurement for the native intact contralateral control limbs (“Native Intact”). FIG. 7B shows the displacement (in mm) of one of the three scaffold-implanted canine cadaver limbs over 100,000 loading cycles, as a representative illustration of the load-displacement of the implanted scaffolds over 100,000 cycles. Load-displacement data for the additional two scaffold-implanted canine cadaver limbs were similar to the data shown in FIG. 7B. The observed maximum displacement for all three samples suggests that the scaffolds described herein are stable up to at least 100,000 cycles—that is, the scaffolds described herein do not demonstrate the type of creep or stretch that might result in clinical failure. The observed maximum displacement further suggests that the scaffolds described herein may benefit from pre-tensioning during implantation to remove the slack responsible for the rise in displacement over the first 10,000+cycles.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1. A scaffold comprising:
a flexible rod having a spiral cross-section, the flexible rod comprising substantially aligned electrospun polymer fibers.

2. The scaffold of claim 1, wherein the flexible rod further comprises substantially uniformly distributed pores.

3. The scaffold of claim 1, wherein the electrospun polymer fibers comprise polyethylene terephthalate and polyurethane in a weight ratio of about 2:8.

4. The scaffold of claim 1, wherein the electrospun polymer fibers comprise at least two electrospun polymer fibers, each electrospun polymer fiber comprising a polymer independently selected from the group consisting of polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, copolymers thereof, and combinations thereof, and wherein the at least two electrospun polymer fibers are co-electrospun.

5. The scaffold of claim 1, wherein the flexible rod has a diameter from about 1 mm to about 25 mm.

6. The scaffold of claim 1, wherein the flexible rod has a length from about 1 cm to about 50 cm.

7. A method of manufacturing a scaffold, the method comprising:
forming a layer of substantially aligned polymer fibers on a mandrel by electro spinning;
rolling the layer from a first end of the mandrel to a second end of the mandrel to form a toroid at the second end of the mandrel; and
cutting the toroid off the mandrel to form a flexible rod having a spiral cross-section.

8. The method of claim 7, wherein the flexible rod further comprises substantially uniformly distributed pores.

9. The method of claim 7, wherein the polymer fibers comprise polyethylene terephthalate and polyurethane in a weight ratio of about 2:8.

10. The method of claim 7, wherein the toroid has a diameter of about 20 cm.

11. The method of claim 7, wherein the layer has a thickness from about 10 μm to about 1,000 μm.

12. The method of claim 7, wherein the flexible rod has a diameter from about 1 mm to about 25 mm.

13. The method of claim 7, wherein the flexible rod has a length from about 1 cm to about 50 CM.

14. A scaffold formed by the process comprising:
forming a layer of substantially aligned polymer fibers on a mandrel by electro spinning;
rolling the layer from a first end of the mandrel to a second end of the mandrel to form a toroid at the second end of the mandrel; and
cutting the toroid off the mandrel to form a flexible rod having a spiral cross-section.

15. The process of claim 14, wherein the flexible rod further comprises substantially uniformly distributed pores.

16. The process of claim 14, wherein the polymer fibers comprise co-electrospinning polyethylene terephthalate and polyurethane in a weight ratio of about 2:8.

17. The process of claim 14, wherein the toroid has a diameter of about 2 cm.

18. The process of claim 14, wherein the layer has a thickness from about 10 μm to about 1,000 μm.

19. The process of claim 14, wherein the flexible rod has a diameter from about 1 mm to about 25 mm.

20. The process of claim 14, wherein the flexible rod has a length from about 1 cm to about 50 cm.
